(19) United States
(12) Patent Application Publication
(10) Pub. No.: US 2015/0342681 A1
(43) Pub. Date: Dec. 3, 2015

(54) SEGMENTED BALLOON LASER ABLATION CATHETER

(71) Applicant: THE SPECTRANETICS CORPORATION, Colorado Springs, CO (US)

(72) Inventor: Weston Lee, Colorado Springs, CO (US)

(21) Appl. No.: 14/700,556

(22) Filed: Apr. 30, 2015

Related U.S. Application Data
(60) Provisional application No. 62/004,686, filed on May 29, 2014.

(51) Int. Cl. A61B 18/24

(52) U.S. Cl. CPC A61B 18/245; A61B 2018/00345

(57) ABSTRACT

The present disclosure relates generally to medical devices, and, in particular, to a system for improved intraluminal positioning of a laser ablation catheter during the removal of material resulting from therapeutic treatment of occlusions within blood vessels. Given the challenges associated with delivering effective therapy for vascular blockages, there remains a need to provide a catheter that can maintain a consistent intraluminal position during the treatment, despite irregularities and inconsistencies within the vessel.

### CROSS-REFERENCE TO RELATED APPLICATION

The present application claims the benefit of and priority to, under 35 U.S.C. §119(e), U.S. Provisional Application Ser. No. 62/004,686, filed May 29, 2014, entitled SEGMENTED BALLOON LASER ABLATION CATHETER, which is hereby incorporated by reference in its entirety for all purposes.

### FIELD

The present disclosure relates generally to medical devices, and, in particular, to a system for improved intraluminal positioning of a laser ablation catheter during the removal of material resulting from therapeutic treatment of occlusions within blood vessels.

### BACKGROUND

Blood vessels often become occluded or blocked by plaque, thrombi, emboli or other deposits which reduce the blood carrying capacity of the vessel and cause damage to the vessel walls. Blockages that occur at critical locations in the vasculature can cause serious and permanent injury, including brain damage, paralysis and death. When blockages are detected, medical intervention is usually necessary to prevent or mitigate these harmful consequences.

Balloon angioplasty and other transluminal medical treatments are well-known and have been proven efficacious in the treatment of stenotic lesions. The application of such medical procedures to certain blood vessels, however, has been limited, due to the risks associated with creation of emboli during the procedure. For example, angioplasty is not the currently preferred treatment for lesions in the carotid artery because of the possibility of dislodging plaque from the lesion, which can enter the various arterial vessels of the brain and cause permanent brain damage. Other types of intervention for blocked vessels include atherectomy, stent insertion, pharmaceutical intervention, and bypass surgery.

One commonly used surgical procedure is a carotid endarterectomy. This procedure involves the surgical removal of plaque from within the carotid artery by creating an incision in the artery and removing the plaque. Carotid endarterectomy may be recommended for patients who have had a transient ischemic attack (TIA) or a mild stroke due to significant carotid artery disease. However, as with all surgical procedures, there is a risk of complications, including bleeding, infection, blood clots, brain damage, stroke or heart attack.

In addition to these challenges, the size of the vessel may limit access to the blockage. Vessels as small as 3 mm in diameter are quite commonly found in the coronary arteries, and even certain saphenous vein graph bypass vessels can also be as small as 3 mm or 4 mm. Other challenges include the wide variety in emboli dimensions and the presence of intraluminal irregularities (e.g., calcium deposits, cholesterol deposits, scar tissue, etc.) that can affect the positioning of the medical devices being used to reduce the blockage. For example, most interventional procedures use a guidewire to aid in the correct positioning of a medical device (e.g., catheter). However, if the blockage is significant, such as with a Chronic Total Occlusion (CTO), or if there are significant intraluminal irregularities or inconsistencies, the guidewire may not be sufficient to establish and maintain a consistent intraluminal position. Additionally, complications involving the guidewire can arise, the most common of which is cardiac arrhythmias; however, other complications include looping and knotting, vascular perforation, fragmentation and embolization, and intravascular entrapment of the wire.

Given the challenges associated with delivering effective therapy for vascular blockages, there remains a need to provide a catheter that can maintain a consistent intraluminal position during the procedure, despite irregularities and inconsistencies within the vessel.

### SUMMARY

These and other needs are addressed by the various aspects, embodiments, and configurations of the present disclosure.

The present disclosure relates generally to medical devices, and, in particular, to a system for improved intraluminal positioning of a laser ablation catheter during the removal of material resulting from therapeutic treatment of occlusions within blood vessels.

In some aspects, a laser ablation catheter of the present disclosure can comprise an inner sheath and an outer sheath, a plurality of optical fibers, a segmented balloon assembly, at least one inflation source coupled to the segmented balloon assembly, and a controller for controlling the inflation of the plurality of balloons according to a predetermined inflation sequence. In some cases, the plurality of optical fibers comprise an area between the inner and outer sheaths, and the inner sheath separates the inner lumen from the plurality of optical fibers.

In some cases, the inner lumen can comprise a port or channel for the insertion of a guidewire or other medical devices. In other cases laser ablation catheter may include a separate or additional lumen that can further comprise other channels or ports for the insertion of other medical devices that can be used in an ablation procedure (e.g., a suction device). The plurality of optical fibers are designed to deliver light energy to the site of an occlusion to facilitate its ablation. The optical fibers can be designed to terminate at the distal end of the catheter. The plurality of optical fibers can be further defined by a separate inner cylindrical wall and a separate outer cylindrical wall, such that the inner lumen and plurality of optical fibers overlap along the longitudinal axis of the catheter body.

In some aspects, a laser ablation catheter of the present disclosure can also comprise a segmented balloon assembly made up of a plurality of balloons or balloon segments. In some cases, the plurality of balloons or balloon segments can be circumferentially aligned around, and be in contact with, the outer sheath or the outer cylindrical wall at the distal end of the catheter. In some cases, the plurality of balloons of the segmented balloon assembly can be functionally engaged such that individual balloons can be inflated and deflated in a sequential manner to rotate the distal end of the catheter in a circular manner. In other cases, the plurality of balloons of the segmented balloon assembly can be functionally engaged such that all the individual balloons can be inflated or deflated simultaneously. In some cases, simultaneous inflation or deflation can establish a certain intraluminal position within the vessel. In some cases, the segmented balloon assembly can comprise 3, 4, 5, 6, 7, 8, 9, 10 or more individual balloons in the assembly, all operating in concert. In other cases, a laser ablation catheter can comprise a plurality of segmented balloon assemblies at its distal end to facilitate the positioning of the catheter in a vessel that contains irregularities and inconsistencies (e.g., calcium deposits, cholesterol deposits, scar tissue, etc.).

In other aspects, a laser ablation catheter of the present disclosure can be used as part of a method of ablating an occlusion. In some cases, the method comprises advancing a guidewire through a proximal entry port of an inner lumen of the catheter, through a distal exit port of the inner lumen of the catheter, and into a region of the occlusion. In some cases, the method further comprises advancing the distal end of the catheter to a working distance from the occlusion, and then establishing an intraluminal position within the vessel by inflating a plurality of balloons in the segmented balloon assembly simultaneously. In some cases, the plurality of balloons can be circumferentially aligned around the distal end of the catheter and be functionally engaged with each other such that, upon activating a rotational sequence, the balloons can be sequentially deflated and inflated and thus advance the catheter through the occlusion. In some cases, the guidewire can be removed after establishing an intraluminal position within the vessel. In other cases, the plurality of balloons in the segmented balloon assembly can be simultaneously inflated after the laser ablation catheter has advanced through a region of the occlusion. In still other cases, the method of the present disclosure can be repeated to facilitate the removal of an occlusion.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view of a distal end of a catheter, according to one embodiment of the present disclosure.

FIG. 2A is a front view of a distal end of a catheter, according to one embodiment of the present disclosure.

FIG. 2B is a cross-sectional view of a distal end of a catheter, according to one embodiment of the present disclosure.

FIG. 3 is a flowchart of a method for operating a catheter, according to one embodiment of the disclosure.

### DETAILED DESCRIPTION

The present disclosure relates generally to medical devices, and, in particular, to a system for improving the intraluminal positioning of a laser ablation catheter during the removal of material resulting from therapeutic treatment of occlusions within blood vessels.

Although a large portion of this disclosure includes a discussion of laser catheters (or catheters having a combination of laser emitters and mechanical cutting instruments at the distal end) used in conjunction with an aspiration system, catheters having mechanical cutting instruments may also be used. Laser catheters typically transmit laser energy through optical fibers housed in a relatively flexible tubular catheter inserted into a body lumen, such as a blood vessel, ureter, fallopian tube, cerebral artery and the like to remove obstructions in the lumen. Catheters used for laser angioplasty and other procedures may have a central passageway or tube which receives a guide wire inserted into the body lumen (e.g., vascular system) prior to catheter introduction. The guide wire facilitates the advancement and placement of the catheter to the selected portion(s) of the body lumen for laser ablation of tissue.

Examples of laser catheters or laser sheaths are sold by the Spectranetics Corporation under the tradenames ELCA™ and Turbo Elite™ (each of which is used for coronary intervention or catheterization such as recanalizing occluded arteries, changing lesion morphology, and facilitating stent placement) and SLSII™ and GlideLight™ (which is used for surgically implanted lead removal). The working (distal) end of a laser catheter typically has a plurality of laser emitters that emit energy and ablate the targeted tissue. The opposite (proximal) end of a laser catheter typically has a fiber optic coupler, which connects to a laser system or generator. One such example of a laser system is the CVX-300 Excimer Laser System, which is also sold by the Spectranetics Corporation.

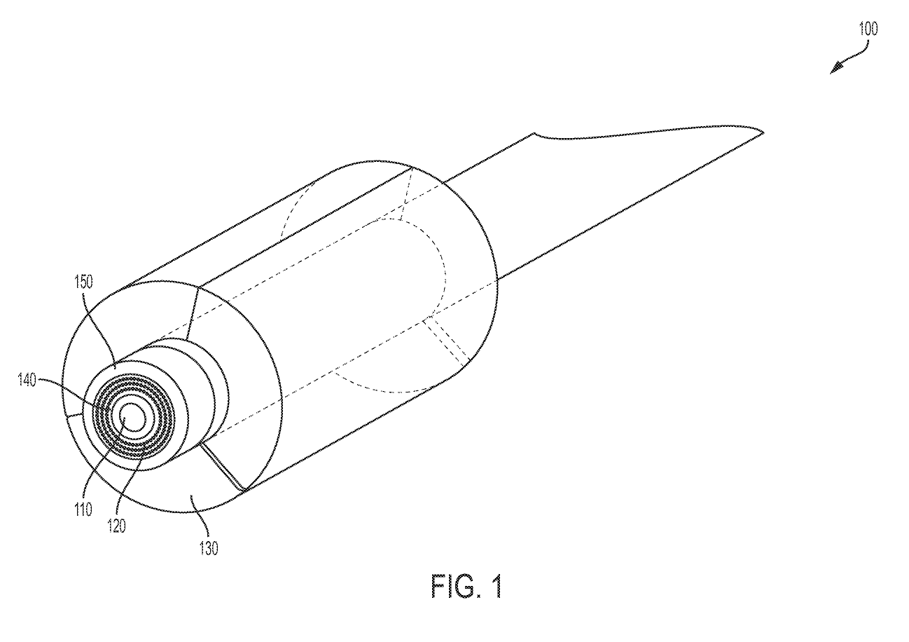

Referring to FIG. 1, the distal end of laser ablation catheter 100 of the present disclosure generally comprises inner lumen 110, plurality of optical fibers 120, and segmented balloon assembly 130. In some embodiments, inner lumen 110 and the plurality of optical fibers 120 can be separated by inner sheath 140, and the plurality of optical fibers 120 can be surrounded by outer sheath 150. Alternatively, the plurality of optical fibers 120 may define the inner cylindrical wall and/or the outer cylindrical wall of the laser ablation catheter 100. In either embodiment, the inner lumen 110 and the plurality of optical fibers 120 overlap along the longitudinal axis of the catheter body.

In some embodiments, inner lumen 110 can comprise a port or channel for the insertion of a guidewire or other medical devices. In other embodiments, inner lumen 110 can include a separate or additional lumen that can further comprise other channels or ports for the insertion of other medical devices that can be used in an ablation procedure (e.g., a suction device). In some embodiments, the plurality of optical fibers 120 is designed to deliver light energy to the site of an occlusion to facilitate its ablation. The plurality of optical fibers 120 can be designed to terminate at the distal end of the catheter. The plurality of optical fibers 120 can be further defined by a separate inner sheath 140 and a separate outer sheath 150, such that the inner lumen 110 and the plurality of optical fibers 120 overlap along the longitudinal axis of the catheter body. In some cases, the plurality of optical fibers 120 can be further defined by a separate inner cylindrical wall and a separate outer cylindrical wall, such that the inner lumen 110 and plurality of optical fibers 120 overlap along the longitudinal axis of the catheter body.

In some embodiments, the laser ablation catheter of the present disclosure can comprise a segmented balloon assembly at its distal end, at least one inflation source that is functionally engaged with and coupled to the plurality of balloons in the segmented balloon assembly, and a controller for activating a predetermined inflation sequence to control the movement of the distal end of the catheter. In some embodiments, the segmented balloon assembly is functionally engaged with and coupled to more than one inflation source. For example, there may be one inflation source for all of the balloons or there be a separate inflation source for each corresponding balloon within the segmented balloon assembly. Regardless, the inflation source or sources can generally be near the proximal end of the laser ablation catheter and the inflation source or sources can be used to transfer an inflation medium (e.g., compressed air, purified air, fluid) from the proximal end of the catheter to the plurality of balloons in the segmented balloon assembly at the distal end of the catheter.

In some embodiments, the inflation source or sources are operated by a controller located at the proximal end of the catheter. In some embodiments, the inflation source or sources can be functionally coupled to the controller, such that medical personnel can use the controller to manipulate the inflation source or sources to inflate and deflate the plurality of balloons in order to control the movement of the distal end of the catheter to, for example, ablate a vascular occlusion. In some embodiments, the controller can be used to activate a predetermined inflation or deflation sequence. In some embodiments, the controller comprises a display and an operational interface to facilitate the precise and accurate execution of a predetermined inflation or deflation sequence. In other embodiments, the controller comprises an operation interface that further comprises input controls for operating the plurality of optical fibers. In some embodiments, the controller is operated according to executable software. The software can comprise operational code instructions for creating and executing various inflation and deflation sequences. In some embodiments, the software for executing various inflation and deflation sequences can be created or modified using a computer, laptop, tablet, or similar device. In some embodiments, the controller is integrally coupled to a laser ablation catheter. In some embodiments, the controller is detachably coupled to a laser ablation catheter.

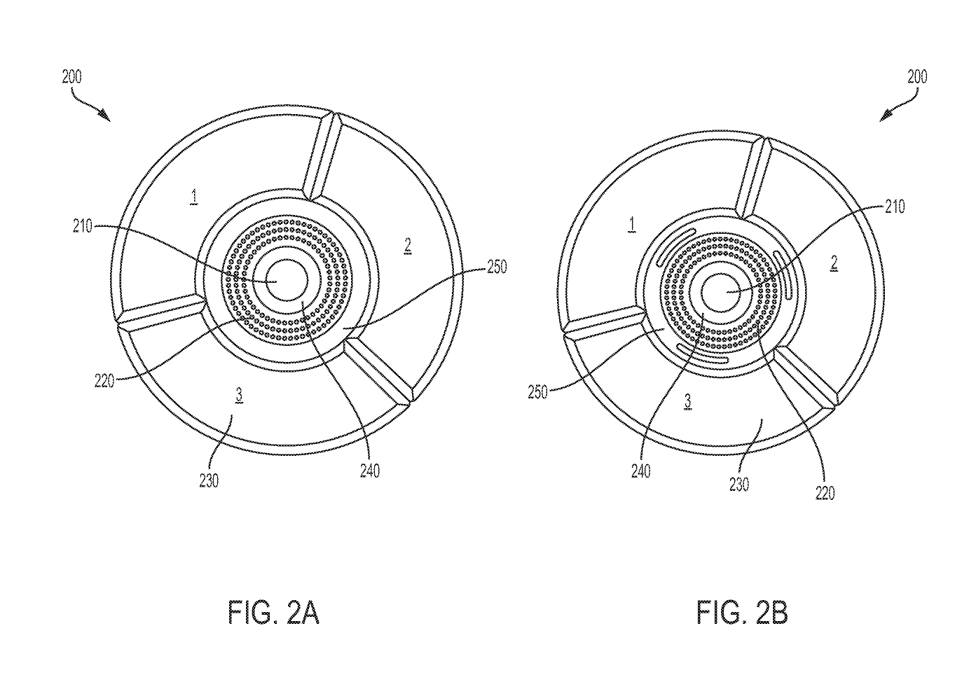

Various catheters designs are contemplated by the present disclosure. Although particular embodiments are shown and described herein, the present disclosure is not so limited. Features of the present disclosure may be provided in combination with various catheter distal end designs. For example, the configuration of the laser emitters of FIGS. 1-2 may arranged such that they extend spirally or helically but in a patter less than 360 degrees. Similarly, the sharp cutting edge or blade may be at an angle or offset from the longitudinal axis of the catheter or it lumen.

Catheter distal tips of the present disclosure include, but are not limited to, purely mechanical cutting devices provided in circular, off-set, and semi-circular arrangements; various combinations of mechanical and laser-ablative cutting systems; and purely laser-ablative cutting systems. For example, the present disclosure can include tips capable of applying laser energy and/or mechanical force (or pressure) to core through occlusive material and create plug-type objects that can be aspirated through the catheter in their entirety. However, certain aspects of this disclosure may be beneficial to various mechanical and/or other types of macerating devices and catheter tips. For example, mechanical tips that may be used to cut and/or macerate lesion-type tissue.

Referring to FIGS. 2A and 2B, the distal end of laser ablation catheter 200 of the present disclosure generally comprises inner lumen 210, a plurality of optical fibers 220, and segmented balloon assembly 230. As shown in the front view of FIG. 2A and the cross-sectional view of FIG. 2B, inner lumen 210 and the plurality of optical fibers 220 can be separated by inner sheath 240, and the plurality of optical fibers 220 can be further defined by outer sheath 250, such that inner lumen 210 and the plurality of optical fibers 220 overlap along the longitudinal axis of the catheter body.

In some embodiments, segmented balloon assembly 230 located at the distal end of laser ablation catheter 200 can comprise a plurality of balloons or balloon segments. In some embodiments, the plurality of balloon can be circumferentially aligned around, and be in contact with, outer sheath 250 of the distal end of laser ablation catheter 200. For example, as shown in FIGS. 2A and 2B, some embodiments of segmented balloon assembly 230 can comprise three balloon segments (e.g., segment 1, segment 2, and segment 3), each of which is functionally coupled to the other two, and each of which is in contact with outer sheath 250, such that the balloon segments occupy the outer circumference of laser ablation catheter 200.

In some embodiments, segmented balloon assembly 230 can comprise three or more individual balloons or balloon segments. In other embodiments, segmented balloon assembly 230 can comprise 4, 5, 6, 7, 8, 9 or 10 or more individual balloon segments. In some embodiments, the balloon segments can essentially be equally sized and shaped. In other embodiments, the balloon segments can be irregularly shaped, with some balloon segments occupying more of the outer circumference of laser ablation catheter 200 than other balloon segments. In some embodiments, the balloon segments can comprise a single segmented balloon assembly 230 located at the distal end of catheter 200, as shown, for example, in FIGS. 2A and 2B. In other embodiments, laser ablation catheter 200 can comprise a plurality of segmented balloon assemblies 230, located at various non-overlapping positions at its distal end.

In some embodiments, laser ablation catheters of the present disclosure can facilitate the positioning of a catheter in a vessel that contains irregularities and inconsistencies (e.g., calcium deposits, cholesterol deposits, scar tissue, etc.). In some embodiments, the plurality of balloons can be functionally engaged such that individual balloon can be inflated and deflated in a sequential manner to rotate the distal end of the catheter in a circular manner. For example, with reference to FIGS. 2A and 2B, all of balloon segments 1, 2, and 3 can be inflated simultaneously and be secured in a certain intraluminal position. Then, balloon segment 1 can be deflated, which will bias the distal end of catheter 200 in a northwesterly direction, viewed from the distal end of the laser ablation catheter 200. Next, balloon segment 1 can be re-inflated, while balloon segment 2 is deflated, which will bias the distal end of the catheter in a northeasterly direction. Next, balloon segment 2 can be re-inflated, while balloon segment 3 is deflated, which will bias the distal end of the catheter in a generally southern direction. In this manner, the distal end of laser ablation catheter will rotate in a clockwise direction, relative to the occlusion.

In some embodiments, the direction of rotation can be counterclockwise. Also, the segmented balloon assembly can be operated such that clockwise and counterclockwise rotations are alternated, depending on the size and shape of the occlusion. Generally, this rotational movement can be accomplished with various numbers of individual balloon segments and various numbers of segmented balloon assemblies functionally coupled and working in concert. The exact numbers of balloons and assemblies may vary depending on the extent of the occlusion and the irregularities present in the area around the occlusion. Generally, as the distal end of the catheter is rotated, the laser can be activated, which will result in the ablation of regions of the occlusion as the distal end of the catheter is rotated. In this general manner, the catheter can be slowly advanced through the vessel, even if the occlusion is significant (e.g., CTO).

In other embodiments, one or more balloon segments can be inflated simultaneously and can be excluded from the rotational sequence of deflation and inflation, such that, for example, the distal end of the catheter may move back and forth in a linear direction corresponding to the inflation and deflation of only two balloon segments. This linear movement may be useful in targeting a specific region of an occlusion or irregularities within the vessel.

In some embodiments, the plurality of balloons of the segmented balloon assembly can be functionally engaged such that all the individual balloons can be inflated or deflated simultaneously. In some embodiments, simultaneous inflation or deflation can establish a certain intraluminal position within the vessel, which will generally be less affected by irregularities and inconsistencies in the vessel (e.g., calcium deposits, cholesterol deposits, scar tissue, etc.). In other embodiments, the presence of the segmented balloon assembly can assure a truer and more accurate intraluminal position because vessel irregularities will not bias the catheter in an unwanted direction or position. For example, medical personnel can operate the segmented balloon assembly such that it targets a specific region or regions of an occlusion without being affected by vessel irregularities.

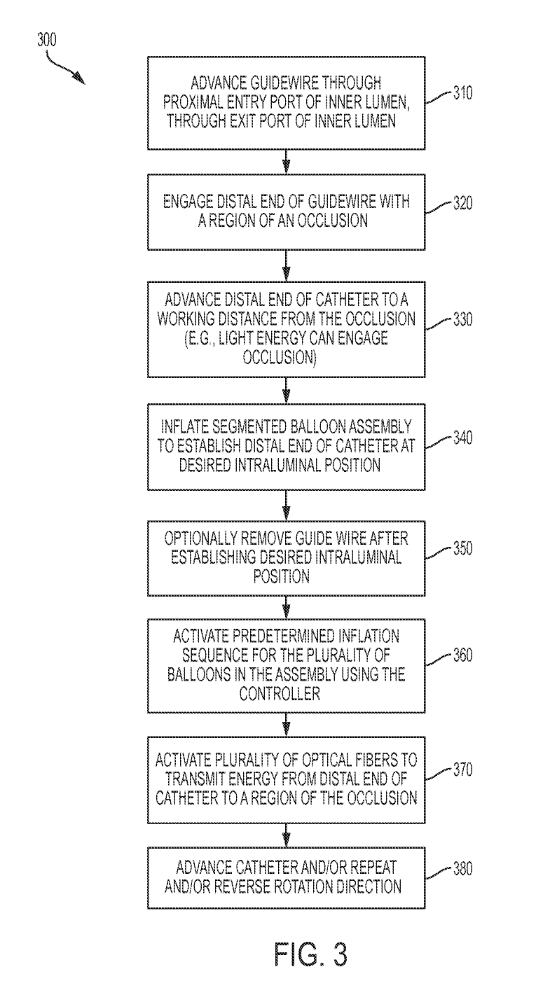

Referring to FIG. 3, a laser ablation catheter of the present disclosure can be used as part of a method of ablating an occlusion in a vessel. For example, at 310, the method can comprise advancing a guidewire through a proximal entry port of an inner lumen of the catheter through a distal exit port of the inner lumen of the catheter, until, at 320, the guidewire engages a region of the occlusion. Next, at 330, the method can comprise advancing the distal end of the catheter to a working distance from the occlusion (e.g., until the light energy delivered from the catheter can affect the occlusion). Next, at 340, the method can comprise establishing a desired intraluminal position within the vessel by inflating a plurality of balloons in the segmented balloon assembly simultaneously. Next, at 350, the method can comprise the optional step of removing the guidewire from the inner lumen of the catheter once the desired intraluminal position is established. Next, at 360, the method can comprise activating a predetermined inflation sequence, wherein the plurality of balloons can be sequentially deflated and inflated to rotate the distal end of the catheter in a clockwise or counterclockwise direction (see, e.g., FIGS. 2A and 2B), or both. Next, at 370, the method can comprise activating, for example, the plurality of optical fibers of the catheter, such that energy is transmitted from the distal end of the catheter to a region of the occlusion. Next, at 380, the method can comprise advancing the distal end of the catheter through the occlusion, or to another region of the occlusion, repeating the rotational sequence, or altering the rotational sequence so that, for example, the direction of rotation is reversed. In some embodiments, after the segmented balloon assembly has passed through an occlusion, the balloon segments can all be inflated simultaneously to expand the hole created in the occlusion, as in an angioplasty procedure.

Embodiments of the present disclosure can also include methods of advancing a laser catheter across an occlusion. The methods may include the steps of advancing a guidewire through a first proximal lumen and a distal lumen of the catheter into a first region of the occlusion, retracting the guidewire from the distal lumen, and advancing a diagnostic device from a second proximal lumen of the catheter to examine the occlusion. The methods may also include retracting the diagnostic device from the distal lumen, and again advancing the guidewire through the distal lumen to advance the guidewire through the occlusion. The laser catheter may be activated to create a lumen through the occlusion. Additional embodiments include alternatively advancing the guidewire and the diagnostic device a plurality of times through the distal lumen to advance the catheter across the occlusion.

Embodiments of the present disclosure still further can include methods of treating an occlusion with a laser catheter. The methods may include the steps of advancing a guidewire through a first proximal lumen and a distal lumen of the catheter into the occlusion, and retracting the guidewire from the distal lumen. The methods may also include illuminating the occlusion with light emitted from a plurality of optical fibers positioned between the distal lumen and an outside surface of the catheter, wherein distal ends of the optical fibers terminate at a distal tip of the catheter. The methods may further include readvancing the guidewire through the distal lumen of the catheter and through the occlusion.

Embodiments of the present disclosure also include catheter assemblies that have a plurality of proximal lumens in a proximal region of the catheter, and a distal lumen in communication with the plurality of proximal lumens. A plurality of optical fibers can reside in the catheter and terminate at a distal tip of the catheter. The assembly may also have multiple diagnostic or therapeutic devices in the proximal lumens, where each device can be selectively advanced into the distal lumen.

Embodiments of the present disclosure can still further include catheter systems having a catheter body with a proximal end and a distal end. The catheter body may include a proximal region having a plurality of proximal lumens that merge into a distal lumen having a cross-sectional area less than a combined cross-sectional area of the proximal lumens. They catheter body may also include a plurality of optical fibers that reside in the catheter body and terminate at the distal end. The systems may also include a laser-assisted guidewire slidably disposed in a first one of the proximal lumens, and a second guidewire slidably disposed in a second one of the proximal lumens. The laser-assisted guidewire may be slidably advanced from the first proximal lumen through the distal lumen to assist in penetrating the occlusion after the second guidewire, which is used to position the catheter at the treatment site, is retracted from the distal lumen to the second proximal lumen.

Embodiments of the present disclosure may also further include catheter assemblies that have catheters with a proximal section and a distal section. The proximal section may have at least two proximal lumens for component devices (e.g., therapeutic, diagnostic and/or steering devices), and at least one additional proximal lumen that holds a light guide (e.g., optical fibers). The distal section may have a first distal lumen for holding component devices, and another distal lumen for holding the light guide. The light guide may run continuously through the proximal and distal lumens that hold the light guide. The catheter assemblies may further include a first wire and a second wire, where at least a portion of the first wire is removably disposed in a first proximal component lumen, and at least a portion of the second wire is removably disposed in the second proximal component lumen. A portion of a selected one of the first wire and the second wire may be removably disposed in the distal lumen for holding component devices. The first wire may be a guide wire, a laser-assisted guide wire, a steering wire, or an imaging wire, among other types of wires and therapeutic devices. The proximal and distal lumens for holding the light guide may have an annular shape.

Embodiments of the present disclosure still also include methods of removing blood clots and other types of occlusions from a blood vessel (e.g., arterial vessels, cerebral vessels, etc.). The methods may include the step of providing a catheter having a proximal section and a distal section. The proximal section may have at least two proximal lumens for component devices (e.g., therapeutic, diagnostic and/or steering devices), and at least one additional proximal lumen that holds a light guide (e.g., optical fibers). The distal section may have a first distal lumen for holding component devices, and another distal lumen for holding the light guide. The light guide may run continuously through the proximal and distal lumens that hold the light guide. The methods may also include inserting a first guide wire having a distal tip into a first proximal component lumen and the distal component lumen such that a portion of the first guide wire is located in the first proximal component lumen, and another portion of the first guide wire is located in the distal component lumen. The catheter and the first guide wire may be advanced to a first treatment site proximate the clot. Energy may be applied to the clot through the light guide. The first guide wire may be withdrawn so that its distal tip is located in the first proximal component lumen. A laser-assisted guide wire having a distal tip and a light guide may be advanced from a second proximal component lumen through the distal component lumen such that a portion of the laser-assisted guide wire is located in the second proximal component lumen, and another portion of the laser-assisted guide wire is located in the distal component lumen. Energy may be applied to the clot through the laser-assisted guide wire light guide.

Embodiments of the present disclosure may yet further include additional methods of removing an occlusion from a patient's blood vessel. The methods may include the step of providing a catheter having a proximal section and a distal section. The proximal section may have at least two proximal lumens for component devices (e.g., therapeutic, diagnostic, and/or steering devices), and at least one additional proximal lumen that holds a light guide (e.g., optical fibers). The distal section may have a first distal lumen for holding component devices, and another distal lumen for holding the light guide. The light guide may run continuously through the proximal and distal lumens that hold the light guide. A guide wire having a distal tip may be inserted into the catheter such that a portion of it is located in a first proximal component lumen and another portion of it is located in the distal component lumen. The catheter and the guide wire may be advanced to a treatment site proximate the occlusion. The guide wire may be withdrawn such that its distal tip is located in the first proximal component lumen. A steering wire having a longitudinal axis and a bent distal section having a distal tip may be inserted into a second proximal component lumen and advanced though the distal component lumen such that a portion of the steering wire is located in the second proximal component lumen, and another portion of the steering wire is located in the distal component lumen. The steering wire may be rotated about the longitudinal axis while energy is applied to the occlusion through the light guide.

Embodiments of the present disclosure may yet further include a laser ablation catheter that includes a plurality of optical fibers defining an outer cylindrical wall and an inner cylindrical lumen, the outer cylindrical wall having a proximal end and a distal end; a segmented balloon assembly comprising a plurality of balloons circumferentially aligned around the distal end of the plurality of optical fibers; at least one inflation lumen coupled to the segmented balloon assembly; at least one inflation source coupled to the segmented balloon assembly; and a controller coupled to the at least one inflation source for controlling the inflation of the plurality of balloons according to a predetermined inflation sequence.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

These and other advantages will be apparent from the disclosure of the aspects, embodiments, and configurations contained herein.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁—Xₙ, Y₁—Yₘ, and Z₁—Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

A “catheter” is a tube that can be inserted into a body cavity, duct, lumen, or vessel, such as the vasculature system. In most uses, a catheter is a relatively thin, flexible tube (“soft” catheter), though in some uses, it may be a larger, solid-less flexible—but possibly still flexible—catheter (“hard” catheter).

A “coupler” or “fiber optic coupler” refers to the optical fiber device with one or more input fibers and one or several output fibers. Fiber couplers are commonly special optical fiber devices with one or more input fibers for distributing optical signals into two or more output fibers. Optical energy is passively split into multiple output signals (fibers), each containing light with properties identical to the original except for reduced amplitude. Fiber couplers have input and output configurations defined as M×N. M is the number of input ports (one or more). N is the number of output ports and is always equal to or greater than M. Fibers can be thermally tapered and fused so that their cores come into intimate contact. This can also be done with polarization-maintaining fibers, leading to polarization-maintaining couplers (PM couplers) or splitters. Some couplers use side-polished fibers, providing access to the fiber core. Couplers can also be made from bulk optics, for example in the form of microlenses and beam splitters, which can be coupled to fibers (“fiber pig-tailed”).

The terms “determine”, “calculate” and “compute,” and variations thereof, as used herein, are used interchangeably and include any type of methodology, process, mathematical operation or technique.

A “laser emitter” refers to an end portion of a fiber or an optical component that emits laser light from a distal end of the catheter towards a desired target, which is typically tissue.

An optical fiber (or laser active fibre) is a flexible, transparent fiber made of an optically transmissive material, such as glass (silica) or plastic, that functions as a waveguide, or “light pipe”, to transmit light between the two ends of the fiber.

The term “computer-readable medium” as used herein refers to any storage and/or transmission medium that participate in providing instructions to a processor for execution. Such a medium is commonly tangible and non-transient/non-transitory and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

A number of variations and modifications of the disclosure can be used. It would be possible to provide for some features of the disclosure without providing others. Furthermore, embodiments of systems and methods according to the present disclosure may include and/or be used in conjunction with any of the systems, devices, structures, and/or methods described in U.S. patent application Ser. Nos. 13/800,651, 13/800,675, 13/800,700, and/or 13/800,728, all of which were filed on Mar. 13, 2013, the disclosures of which are hereby incorporated by reference in their entireties.

As another example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a non-transient/non-transitory storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The present disclosure, in various aspects, embodiments, and configurations, includes components, methods, processes, systems and/or apparatus substantially as depicted and described herein, including various aspects, embodiments, configurations, subcombinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A laser ablation catheter comprising:
an inner sheath and an outer sheath;
a plurality of optical fibers, wherein the plurality of optical fibers comprise an area between the inner sheath and the outer sheath;
an inner lumen, wherein the inner sheath separates the inner lumen from the plurality of optical fibers;
a segmented balloon assembly comprising a plurality of balloons circumferentially aligned around the outer sheath at the distal end of the catheter;
at least one inflation source coupled to the segmented balloon assembly; and
a controller for controlling the inflation of the plurality of balloons according to a predetermined inflation sequence.

2. The laser ablation catheter of claim 1, wherein the inner lumen further comprises a suction channel for removing occlusive fragments during an ablation procedure.

3. The laser ablation catheter of claim 1, wherein the plurality of optical fibers terminate at the distal end of the catheter.

4. The laser ablation catheter of claim 1, wherein the plurality of balloons are in contact with the outer sheath.

5. The laser ablation catheter of claim 1, wherein the plurality of balloons are functionally engaged with each other and with at least one inflation source to rotate the distal end of the catheter.

6. The laser ablation catheter of claim 1, wherein the plurality of balloons are functionally engaged with each other and with at least one inflation source to inflate or deflate simultaneously.

7. The laser ablation catheter of claim 1, wherein the segmented balloon assembly and the controller are functionally coupled.

8. The laser ablation catheter of claim 1, wherein the segmented balloon assembly comprises 3, 4, 5, 6, 7, 8, 9, 10 or more individual balloons functionally engaged to operate during an ablation procedure.

9. A segmented balloon assembly for a laser ablation catheter comprising:
a plurality of balloons circumferentially aligned around the distal end of the catheter,
wherein the plurality of balloons are functionally engaged with each other and with at least one inflation source such that the plurality of balloons sequentially inflate and deflate during a laser ablation procedure to produce rotational movement.

10. The segmented balloon assembly of claim 9, wherein the plurality of balloons are in contact with the outer wall of the catheter.

11. The segmented balloon assembly of claim 9, wherein the plurality of balloons are functionally engaged with each other and with at least one inflation source to inflate or deflate simultaneously.

12. The segmented balloon assembly of claim 9, wherein the segmented balloon assembly comprises 3, 4, 5, 6, 7, 8, 9, 10 or more individual balloons functionally engaged to operate during an ablation procedure.

13. The segmented balloon assembly of claim 9, wherein the segmented balloon assembly is functionally coupled to a controller for controlling the inflation of the plurality of balloons according to a predetermined inflation sequence.

14. A method of engaging an occlusion in a vessel using a segmented balloon assembly on the distal end of a laser ablation catheter, the method comprising:
advancing a guidewire through a proximal entry port of an inner lumen of the catheter through a distal exit port of the inner lumen of the catheter, and into a region of the occlusion;
advancing the distal end of the catheter to a working distance from the occlusion;
establishing an intraluminal position within the vessel by inflating a plurality of balloons in the segmented balloon assembly simultaneously, wherein the plurality of balloons are circumferentially aligned around the distal end of the catheter;
using a controller to activate a predetermined inflation sequence to sequentially deflate and inflate individual balloons within the segmented balloon assembly to move the catheter; and
activating a plurality of optical fibers to transmit energy from the distal end of the catheter to ablate a region of the occlusion.

15. The method of claim 14, wherein the guidewire is removed after establishing an intraluminal position within the vessel.

16. The method of claim 14, further comprising simultaneously inflating the plurality of balloons in the segmented balloon assembly after the laser catheter has advanced through a region of the occlusion.

17. The method of claim 14, wherein the plurality of balloons are in contact with the outer wall of the distal end of the catheter.

18. The method of claim 14, wherein the plurality of balloons are functionally engaged with each other and with at least one inflation source to inflate and deflate sequentially to produce rotational movement.

19. The method of claim 14, wherein the segmented balloon assembly is functionally coupled to the controller for controlling the inflation of the plurality of balloons according to the predetermined inflation sequence.

20. The method of claim 14, wherein the segmented balloon assembly comprises 3, 4, 5, 6, 7, 8, 9, 10 or more individual balloons functionally engaged to operate during an ablation procedure.
